(19) United States
(12) Patent Application Publication
(10) Pub. No.: US 2025/0160891 A1
(43) Pub. Date: May 22, 2025

(54) OBTURATOR WITH STIFF DISTAL CANNULA ENGAGEMENT REGION

(71) Applicant: Neptune Medical Inc., Burlingame, CA (US)

(72) Inventors: Garrett J. GOMES, Pleasant Hill, CA (US); Kendall CARPENTER, Redwood City, CA (US)

(21) Appl. No.: 18/723,413

(22) PCT Filed: Dec. 21, 2022
(86) PCT No.: PCT/US2022/082141

§ 371 Date: Jun. 21, 2024

Related U.S. Application Data
(60) Provisional application No. 63/292,415, filed on Dec. 21, 2021.

(51) Int. Cl. A61B 17/34; A61M 25/01

(52) U.S. Cl. CPC A61B 17/3496; A61M 25/0102

(57) ABSTRACT

Disclosed herein are obturators that prevent or reduce gaps between the obturator and a catheter in which the obturator is inserted when bending the combined obturator and catheter. For example, described herein are obturators including a distal cannula engagement region that is configured to match the stiffness of the distal end region of the catheter into which the obturator is inserted. Alto described herein are obturator including a distal cannula engagement region having a stiffness that is greater than that of surrounding portions of the obturator.

### CROSS REFERENCE TO RELATED APPLICATIONS

This patent application claims priority to U.S. provisional patent application No. 63/292,415, filed on Dec. 21, 2021, and titled “OBTURATOR WITH STIFF DISTAL CANNULA ENGAGEMENT REGION”, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This application relates to the field of obturators for use in medical procedures.

### BACKGROUND

An obturator is a medical device that may be used to center and lead a catheter as it is guided along a guidewire to target anatomy. For example, obturators may be used as part of a trocar assembly (e.g., including a cannula and an obturator) for laparoscopic, endoscopic or arthroscopic surgery, or virtually any surgical procedure in which a cannulated member (e.g., cannula) is inserted into the body. Obturators typically extend beyond the cannula into which they are inserted and may have a tapered or even pointed distal end region that may help with insertion into the body. In some cases the obturator may be solid; alternatively an obturator may be hollow and/or may include one or more elongated lumen therethrough.

It has long been recognized that in some instances, particularly when navigating tortuous portions of the anatomy, a gap may form between the cannula and the outer surface of the obturator. The presence of a gap between a catheter and an obturator is known as tip gap. When this gap is the result of a catheter and an obturator used in curved anatomy, gaps can be accentuated and become known as fish-mouthing. These types of gaps are combined together and are referred to simply as ‘gap’ or ‘gaps’.

Gaps can be a patient safety concern and/or impact the functionality and trackability of the catheter through a vessel. Gaps can lead to pinching of tissue, which can cause patient trauma and procedural complications. Gaps can catch on branching vessels, impeding advancement through the anatomy. There is a need for a device or system for minimizing gaps between a catheter and an obturator.

### SUMMARY OF THE DISCLOSURE

Described herein are methods and apparatuses (e.g., systems, devices, including obturators) that may provide obturators that prevent gap (fish-mouthing) between the obturator and a cannula. In particular, described herein are methods and apparatuses in which at least a distal end region of the obturator that is configured to align with a distal end region of the cannula (e.g., including the distal end opening of the cannula), referred to herein as the distal cannula engagement region of the obturator, has a stiffness that is greater than a stiffness of regions of the obturator that are immediately proximal and/or distal to the distal cannula engagement region.

Gaps are typically created when there is a large difference in bending stiffness between the obturator and distal end region of the cannula. The gap is accentuated when the distal tip of the distal end region is made of a low durometer low stiffness material and the region just proximal to the distal tip is stiffer than the distal tip. The distal tip is often made to have a low durometer and low stiffness in order to not scrap or damage anatomy during the procedure. The region just proximal to the distal tip is typically stiffer than the distal tip since the body of the cannula must retain its shape, withstand pushing, pulling, torsion, and pinching loads. The region just proximal to the distal tip is also typically stiffer than the distal tip because reinforcing elements such as wires, braids, and lubricious liners terminate in this region and those terminations require reinforcements such as welds, adhesives, and heat shrink to keep the reinforcing elements from detaching. When the distal tip is lower stiffness than the region just proximal to the distal tip, bending loads from the obturator are applied to the distal tip and rather than bending the stiffer proximal region, they deform the soft distal tip, ovalizing the distal tip and creating a gap. As used herein the distal end region of the catheter may refer to the generally stiffer distal end region of the catheter (e.g., the region just proximal to the distal tip).

The stiffness of a catheter and/or an obturator may be measured in terms of flexural modulus. In general, flexural modulus is the measure of a material's bending stress relative to elongation under load. It provides an elastic measure of a material's stiffness for a given test specimen and shape. For example, the rigidity of a polymer tube may be determined by the inherent stiffness of the material (modulus) and the cross sectional design of the catheter. Changing the dimensions of the cross section can have a profound impact on rigidity. As described herein, when describing the relative stiffness of the catheter or obturator, the flexural modulus may be used. The flexural modulus may be described in pascals (Pa or N/m² or m⁻¹·kg·s⁻²); in practice the units may be megapascals (MPa or N/mm²) or gigapascals (GPa or kN/mm²).

In some examples, the stiffness of the distal cannula engagement region of the obturator may be greater than a percentage of the stiffness of the region of the obturator immediately adjacent to the distal cannula engagement region. For example the stiffness of the distal cannula engagement region of the obturator may be 10% or greater (e.g., 12.5% or greater, 15% or greater, 17.5% or greater, 20% or greater, 22.5% or greater, 25% or greater, 27.5% or greater, 30% or greater, 40% or greater, 45% or greater, 50% or greater, etc.) of the stiffness of the region immediately adjacent to the distal cannula engagement region of the obturator, either proximally and/or distally.

Also described herein are obturators having a distal end region (distal cannula engagement region) that matches the stiffness of the cannula, and in particular the distal end region of the cannula. Thus, any of the obturators described herein may have a distal cannula engagement region of the obturator that has a stiffness matching the stiffness of the distal end region of the cannula in which the obturator is inserted. The stiffness-matched distal cannula engagement region of the obturator may be matched to the stiffness of the distal end region of the cannula within +/−a percentage of the stiffness of the distal end region of the cannula. For example, the stiffness may be matched to within +/−20%, +/−15%, +/−14%, +/−13%, +/−12%, +/−11%, +/−10%, +/−9%, +/−8%, +/−7%, +/−6%, +/−5%, +/−4%, +/−3%, +/−2%, +/−1%, etc. The regions of the obturator distal or proximal to the stiffness-matched distal cannula engagement region of the obturator may be significantly more or less stiff than the distal end region of the cannula. In some examples the entire length of the obturator may be matched to the stiffness of the distal end region of the cannula, or just a portion of the obturator that is configured to align with the distal end region of the cannula may be stiffness matched.

When matching the distal end region (e.g., distal cannula engagement region) of the obturator to the stiffness of the cannula, the stiffness may be matched to the stiffness of the region just proximal to the distal tip, which is typically stiffer than the distal tip. In general, the stiffness of the distal end region (e.g., distal cannula engagement region) of the obturator may be matched to the stiffness of the stiffest region of the distal end region of the cannula (e.g., the stiffest region of the distal x mm (e.g., distal 2 mm, 3 mm, 4 mm, 5 mm, 6 mm, 7 mm, 8 mm, 9 mm, 10 mm, etc.).

The obturators described herein may have both a distal cannula engagement region having a stiffness that is greater than a stiffness of the regions of the obturator that are immediately proximal and/or distal to the distal cannula engagement region and may also have a stiffness of the distal cannula engagement region that is matched to the stiffness of the distal end region of the cannula (e.g., within +/−a percentage of the stiffness of the distal end region of the cannula).

In general, the distal cannula engagement region of the obturator may extend between 1 mm and 40 mm along the length of a distal end region of the obturator. For example, the distal cannula engagement region may extend a distance of 2 mm or more (e.g., 3 mm or more, 4 mm or more, 5 mm or more, 6 mm or more, 7 mm or more, 8 mm or more, 9 mm or more, 10 mm or more, 11 mm or more, 12 mm or more, 13 mm or more, 14 mm or more, 15 mm or more, between 2-40 mm, between 2-35 mm, between 2-30 mm, between 2-25 mm, between 2-20 mm, between 2-15 mm, between 2-10 mm, between 2-5 mm, etc.). The majority of the length of the obturator, including regions proximal to the distal cannula engagement region and/or distal to the distal cannula engagement region may have a different stiffness than the distal cannula engagement region (e.g., may be less stiff than the distal cannula engagement region of the cannula).

The distal cannula engagement region of the obturator may be made stiffer than (or in some examples, may be made to match the stiffness of the distal end region of the cannula) by including an insert within the body of the obturator in the region distal cannula engagement region that is configured to be aligned with the distal end region of the cannula in use (e.g., when navigation through the body). For example, the distal cannula engagement region of the obturator may include an internal tubular member having a stiffness that is greater than other regions of the obturator (and/or may match the stiffness of the distal end region of a cannula into which the obturator is to be inserted).

In some examples the distal cannula engagement region of the obturator is formed of a material that has a stiffness (flexural modulus) that is greater than the stiffness of other regions of the obturator (or in some examples, has a stiffness that matches the stiffness of the distal end region of the cannula, e.g., within +/−a percentage of the stiffness of the distal end region of the cannula).

The distal cannula engagement region (or in some variations, the insert within the distal cannula engagement region), may be formed of a harder plastic material, so that it does not flex or deflect as much as the tapered distal trip region of the obturator and/or the adjacent proximal region of the obturator. The bulk of the obturator may be elastomeric, so that it more easily flexes. Elastomers may be measured by hardness (durometer), though there is an approximate relationship between durometer and modulus for elastomers. See, e.g., Dow Corning White Paper, “Can You Estimate Modulus From Durometer Hardness for Silicones?”, at https://www.dow.com/content/dam/dcc/documents/en-us/tech-art/11/11-37/11-3716-01-durometer-hardness-for-silicones.pdf, herein incorporated by reference in its entirety).

For example, described herein are systems comprising: a catheter having a distal end region having a first stiffness; an obturator having a distal cannula engagement region having a stiffness that is matched to the first stiffness, wherein the stiffness of the distal cannula engagement region is different from the stiffness of a tapered region distal to the distal cannula engagement region and is also different from a region of the obturator proximally adjacent to the distal cannula engagement region; wherein the obturator and catheter are configured to engage to form a combined structure with each other proximally so that the distal cannula engagement region is aligned with the distal end region of the catheter to prevent forming a gap between the obturator and the catheter when the combined structure is bent or curved.

The distal cannula engagement region may comprise a stiffening component. The stiffening component may comprise a tubular member within the distal cannula engagement region. The stiffening component may comprise a material having a higher stiffness than a material forming the tapered region distal and/or the region of the obturator proximally adjacent to the distal cannula engagement region. In some examples the stiffening component comprises an internal frame.

The distal cannula engagement region may comprise a length that is between 1 mm and 40 mm. For example, the distal cannula engagement region may comprise a length that is greater than 5 mm.

The obturator may comprise an elongate guidewire channel extending down a length of the obturator.

In some examples the stiffness that matched to the first stiffness is within +/−5% of the first stiffness. The distal cannula engagement region may comprise a stiffening insert within the distal cannula engagement region.

For example, an obturator may include: an elongate body comprising a tapered distal end and comprising a first material; a distal cannula engagement region of the elongate body, the distal cannula engagement region including an insert, wherein the distal cannula engagement region is proximal to the tapered distal end of the elongate body and distally adjacent to a second region of the elongate body, the insert comprising a material having a stiffness that is greater than the stiffness of the first material so that the stiffness of the distal cannula engagement region is greater than the stiffness of the tapered distal end and the second region.

The obturator may include an obturator lumen extending through the elongate body. The insert may comprise an insert lumen coaxial with the obturator lumen. The insert may have a length of between about 1 mm-20 mm. The insert may have a stiffness of between about 0.2 and about 2.5 GPa. In some examples the stiffness of the insert is greater than twice the stiffness of the first material. The insert may be coaxial with the obturator. The obturator may comprise an obturator or a dilator.

The insert may have an outer diameter that is less than the outer diameter of the distal cannula engagement region.

Any of the obturators described herein may include a stop on the proximal end region of the obturator configured to secure the distal cannula engagement region of the obturator in alignment with a distal end region of a catheter in which the obturator is inserted.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A shows an example of an obturator including a distal cannula engagement region.

FIG. 1B shows an example of a cannula.

FIG. 1C shows the obturator of FIG. 1A engaged with the cannula of FIG. 1B so that the distal cannula engagement region is engaged with the distal end region of the cannula.

FIGS. 2A and 2B show an example of gapping (e.g., fish mouthing) occurring between an obturator and a catheter.

FIG. 3 shows an embodiment of an obturator including a stiffening component in the distal cannula engagement region.

FIG. 4 shows a comparison of obturator and catheter systems with and without a stiffening component like that shown in FIG. 3.

FIG. 5A illustrates an obturator having a distal cannula engagement region, which is matched to the stiffness of the distal end region of the catheter within which the obturator is inserted.

FIG. 5B shows an example of a proximal end an obturator including a distal cannula engagement region.

FIGS. 6A-6B show an example of an obturator that includes a distal cannula engagement region having two stiffness-matched regions arranged in-line having different stiffnesses, which may be used with catheters having different stiffness in their distal end regions. FIG. 6A shows the obturator in a first position, with the first stiffness-matched region aligned with a distal end region of a first catheter (having an equivalent or nearly-equivalent stiffness). FIG. 6B shows an obturator in a second position with the second stiffness-matched region aligned with a distal end region of a second catheter.

### DETAILED DESCRIPTION

In general, described herein are apparatuses (devices and systems, including obturators) and methods for using them that prevent or reduce “fish-mouthing” or gaps between the obturator and a catheter within which the obturator is positioned as the two are navigated through a curved body lumen. These apparatuses may include obturators that have a distal cannula engaging region that is configured to have stiffness (flexural modulus) that is approximately the same as the stiffness of the distal end region of the cannula into which the obturator is positioned. In some examples, the obturator had a distal cannula engaging region that is stiffer than other adjacent regions of the obturator. For example, the obturator may include an insert that is positioned within the distal cannula engaging region. For example, insert may be positioned proximal to the tapered distal end of the elongate body of the obturator. The insert may include a material having a stiffness that is greater than the stiffness of adjacent region of the obturator, which may be formed of a less stiff material.

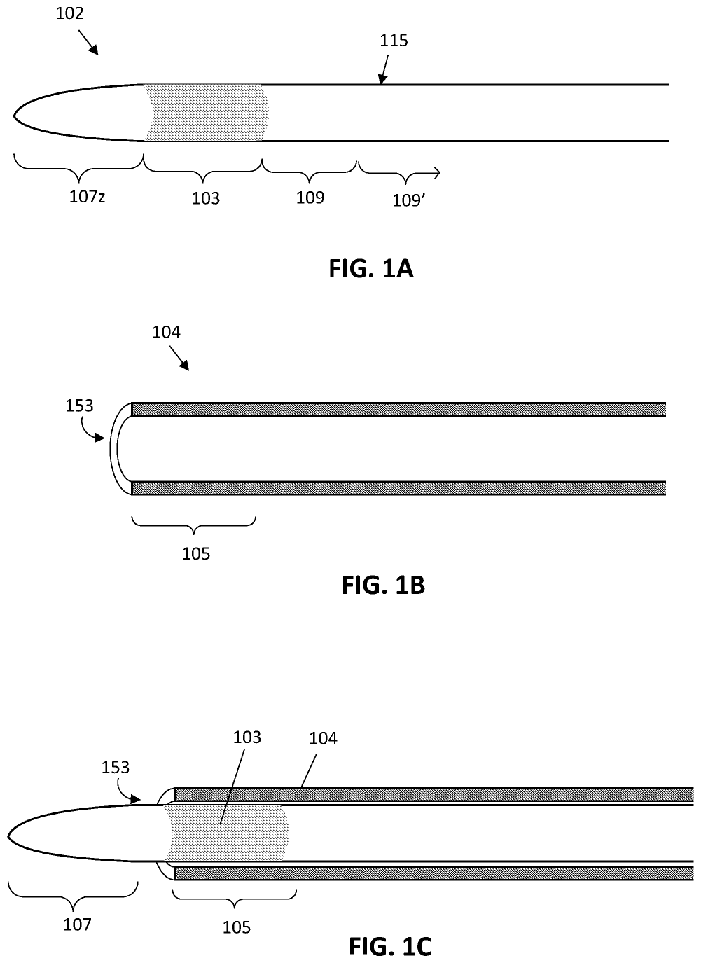

For example, FIG. 1A illustrates an example of an obturator 102. The obturator has an elongate body 115 and a tapered distal end 107. The obturators described herein also include a distal cannula engaging region 103 that is proximal from the tapered distal end and is configured to engage with and the distal end region of a cannula (see, e.g., FIG. 1C), so that the distal cannula engaging region 103 may span the distal end opening of the distal end region.

The obturator may be solid or hollow. In some examples the obturator includes a lumen extending through the length of the obturator.

In FIG. 1A, the distal cannula engagement region 103 may be stiffer than the adjacent distal region (which may include the tapered distal end 107) and the proximal body region 109, 109′ of the elongate member 115. In some cases the distal cannula reengagement region is formed of a stiffer material than the adjacent regions. In some cases the distal cannula region includes an internal structure or insert (e.g., support, frame, tube, etc.) that increases the stiffness of the distal cannula reengagement region. The outer diameter of the obturator may be the same along the obturator until the tapered distal end 107. The outer diameter of the obturator may generally be less than the inner diameter of the cannula into which the obturator is to be inserted. The insert may be the full diameter, or it may be a portion of the diameter (as described in reference to FIG. 3, below).

The obturator may be used with a cannula 104 such as the cannula shown in FIG. 1B. The cannula includes a distal end region 105 that includes a distal end opening 153. Any appropriate cannula may be used and may be configured to engage with the obturator so that the obturator and the cannula engage with each other, as shown in FIG. 1C, with the distal cannula engagement region 103 aligned with the distal end region 105 of the catheter. In this example the distal cannula engagement region extends slightly from the distal end opening 153 of the catheter. In some examples the distal cannula engagement region is configured to align with the distal end opening. The cannula and obturator may be configured to engage with each other, e.g., at the proximal ends of the cannula and obturator, so that the obturator is held aligned with the distal end region, as shown in FIG. 1C.

The distal cannula engagement region (and therefore the stiffness matching region or stiffer region) may extend for any appropriate length of the obturator, e.g., between 1 mm and 50 mm (e.g., between 1 mm and 40 mm, between 2 mm and 40 mm, between 1 mm and 35 mm, between 2 mm and 35 mm, between 1 mm and 30 mm, between 2 mm and 30 mm, between 1 mm and 25 mm, between 2 mm and 25 mm, etc.). Similarly, the distal end region of the catheter may extend for any appropriate length of the catheter, and typically reflect the stiffness of the distal end. The distal end region of the catheter may be shorter than the distal cannula engagement region. For example, the distal end region of the catheter may be between 1 mm and 50 mm, between 1 mm and 45 mm, between 1 mm and 40 mm, between 1 mm and 35 mm, between 1 mm and 30 mm, between 1 mm and 25 mm, between 1 mm and 20 mm, between 1 mm and 15 mm, between 1 mm and 10 mm, between 1 mm and 9 mm, between 1 mm and 8 mm, between 1 mm and 7 mm, between 1 mm and 6 mm, between 1 mm and 5 mm, between 1 mm and 4 mm, between 1 mm and 3 mm, between 1 mm and 2 mm, etc.).

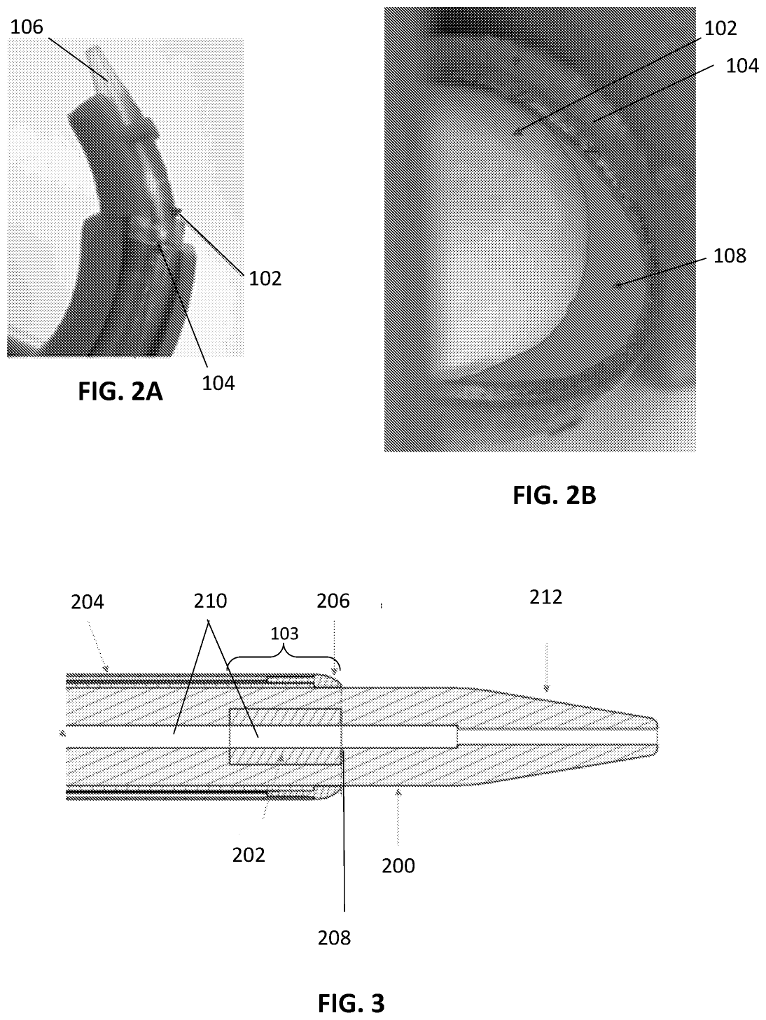

Referring to FIGS. 2A and 2B, an obturator 102 is shown positioned within a catheter 104. The obturator tip 106 is shown deflected (e.g., bent), showing the gap 108 between the obturator tip 106 and the catheter. The gap 108 is shown clearly in the enlarged view of FIG. 2B. Thus, when an obturator such as the one shown in FIGS. 2A-2B is navigated over a bent pathway, the obturator and the distal end opening of the catheter distal end region may separate resulting in gaps that may snag or tear the walls of the body.

Gaps such as that shown in FIGS. 2A and 2B can be exacerbated when there are stiffness mis-matches between components. Catheters are often configured to be very flexible, particularly in the distal end region of the catheter, and can be more or less flexible than an obturator. A catheter tip can be of varying stiffness, including plastic catheter tips (comparatively rigid) and elastomeric tips (comparatively soft, flexible, and deformable). Catheter tips are thin-walled structures, which may allow them to be easily deflected.

Obturators may be solid bodies (or may include a narrow channel, e.g., for a guidewire), which may exhibit a certain stiffness as a result of that geometry. An obturator tip can be very flexible at its distal most tapered-down point, so that it easily tracks on a guidewire. However, proximal to this distal-most point, the obturator tip can widen proximally to a generally constant diameter section in which its geometry creates a stiffer body. In some examples, the obturator may vary in diameter to minimize the potential tip gap distance.

Stiffness mis-matches can include instances when the catheter's tip is softer and/or more flexible than the catheter body and the obturator has a flexible distal end. The increased flexibility and/or softness of one component can allow it to deflect more than a more rigid and/or harder component under the same application of force. This difference in amount of deflection can lead to gapping.

In some embodiments, the obturator can be more flexible than the catheter. In some embodiments, the catheter can be more flexible than the obturator.

Disclosed herein are embodiments of obturators comprising a comparatively rigid distal cannula engagement region of the obturator (e.g., in some cases including a stiffening insert) in proximity to the axial location where there is a point of contact between the catheter tip and the obturator. The distal cannula engagement region may be configured to substantially match the stiffness of the distal end region of the cannula 104 in which the obturator 102 is inserted. More closely matching stiffnesses or flexibilities of the catheter tip and the obturator can help to minimize gaps.

FIG. 3 shows an embodiment of an obturator 200 comprising a distal cannula engagement region 103 including an internal stiffening component 202. The obturator 200 is shown positioned within a catheter 204. The distal end 206 of the catheter can be aligned with the distal cannula engagement region 203 (and in this example, the distal end 208 of the stiffening component 202). In this example, the obturator and the stiffening component both comprise a bore or lumen 210 allowing passage of a guidewire.

The inset 202 shown in FIG. 3 is one example of a stiffening component. The insert may have an outer diameter that is a portion of the diameter of the distal cannula engagement region, or it can be the full diameter of the distal cannula engagement region. The length of the insert may be the full length of the distal cannula engagement region or the length of the insert may be a portion of the distal cannula engagement region. For example, the length may be between about 2 mm and about 20 mm, between about 2 mm and about 17.5 mm, between about 2 mm and about 15 mm, between about 2 and about 12.5 mm, between about 2 mm and about 10 mm, etc. In some examples multiple inserts may be included in the distal cannula engagement region. The insert may be formed of a stiffer material, such as a higher durometer material, e.g., an elastomer or a plastic having a Shore A durometer of between about 80-110 (e.g., Shore A 80, 85, 90, 95, 100, 105, etc.) or a Shore D durometer of between about 20-100 (e.g., Shore D 20, 25, 30, 35, 40, 45, 50, 55, 60, 65, 70, 75, 80, 85, 90, 95, etc. or any sub-region therein). In some examples the insert may have a modulus of 0.2 GPa or greater. In general, and as shown in FIG. 3, the distal end region of the cannula may have two (or more) sections. For example, the distal end region may include a distal tip 206, and a bond region just proximal to the distal tip, bonding the distal tip, which may be a lower durometer (e.g., softer) material, to the rest of the cannula.

The distal cannula engagement region, and in some examples the stiffening component, can have a variety of different diameters, lengths, and may be made of a variety of different materials. In some embodiments, the inserts can have lengths of about 2 mm and 40 mm (e.g., between about 2 mm and 30 mm, between about 2 mm and 25 mm, between about 2 mm and 20 mm, between about 2 mm and 15 mm, between about 2 mm and 10 mm, between about 2 mm and 8 mm, etc.).

In some embodiments, the stiffening component can comprise a cylindrical shape. Other configurations are also possible (e.g., rectangular, tubular, etc.). In some examples the stiffening component may be a mesh material. In some examples the distal cannula engagement region may be formed of a more rigid material, including a more rigid polymeric material. In some examples the distal cannula engagement region may include a support or frame material within the distal cannula engagement region.

Dimensions of the stiffening component can be selected based on the desired rigidity of the component. The desired rigidity can depend on the stiffness match between the catheter and the obturator. As mentioned above, in any of these examples the distal cannula engagement region stiffness may be matched to the stiffness of the distal end region of the cannula. For example, the stiffness of the distal cannula engagement region may be within +/−a percentage of the stiffness of the distal end region of the catheter. For example, the stiffness of the distal cannula engagement region may be matched to within +/−20%, +/−15%, +/−14%, +/−13%, +/−12%, +/−11%, +/−10%, +/−9%, +/−8%, +/−7%, +/−6%, +/−5%, +/−4%, +/−3%, +/−2%, +/−1%, etc. of the distal end region of the cannula. In variations including a stiffening component, the rigidity of the obturator in the distal cannula engagement region may be the combination of the rigidity of the stiffening component and the surrounding obturator material and structure. The stiffening component may be axially aligned with device body or may favor one side if intended curves are known.

In general, the obturator may be formed of a rigid material, such as a plastic (e.g., polycarbonate) or a high durometer elastomer (>Shore A 100). Examples of these materials may include but are not limited to ABS (Acrylonitrile Butadiene Styrene), ASA (Acrylonitrile Styrene Acrylate), ETFE (Ethylene Tetrafluoroethylene), LDPE (Low Density Polyethylene), polyamides, PET (Polyethylene Terephthalate), etc.

The distal cannula engagement region can be formed in any appropriate manner and may include adding a stiffening component. For example, the distal cannula engagement region can be formed by: overmolding a soft material over a rigid material, butt-joining a rigid material between two softer materials, adding a ring of rigid material to the outside of a softer material, and injecting a different material (e.g., a UV-cure adhesive) into a cavity within the softer body. The subject device could be another catheter instead of an obturator.

In any of the apparatuses described herein, more closely matching the stiffnesses between a catheter and obturator at a point of potential gapping can allow for softer catheter tips, which can minimize trauma to the patient. More closely matching the stiffnesses can also help to minimize gaps, providing many clinical advantages, including reduced procedural complications.

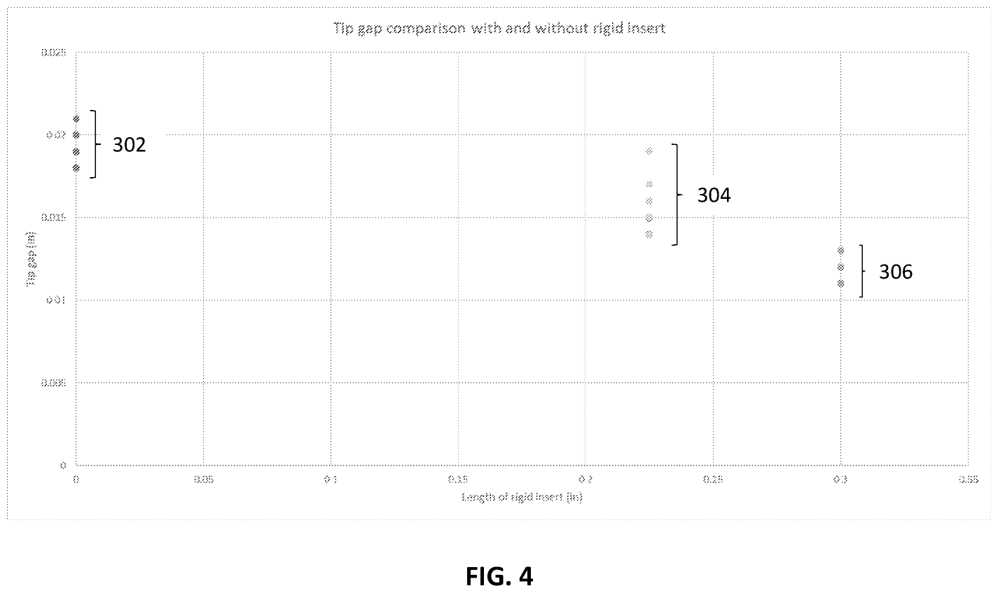

FIG. 4 shows a comparison of tip gaps present in a system with and without a stiffening component (e.g., with and without matching the stiffness of the distal cannula engagement region and the stiffness of the catheter distal end). In this example the obturator is similar to that shown including a stiffening component in FIG. 3. The points 302 represent a system with no stiffening component. The data points 304 represent a system with a stiffening component with a length of 5.84 mm. The data points 306 represent a system with a stiffening component with a length of 7.62 mm. As shown by this data, the gaps were approximately 40% higher for a system without an insert.

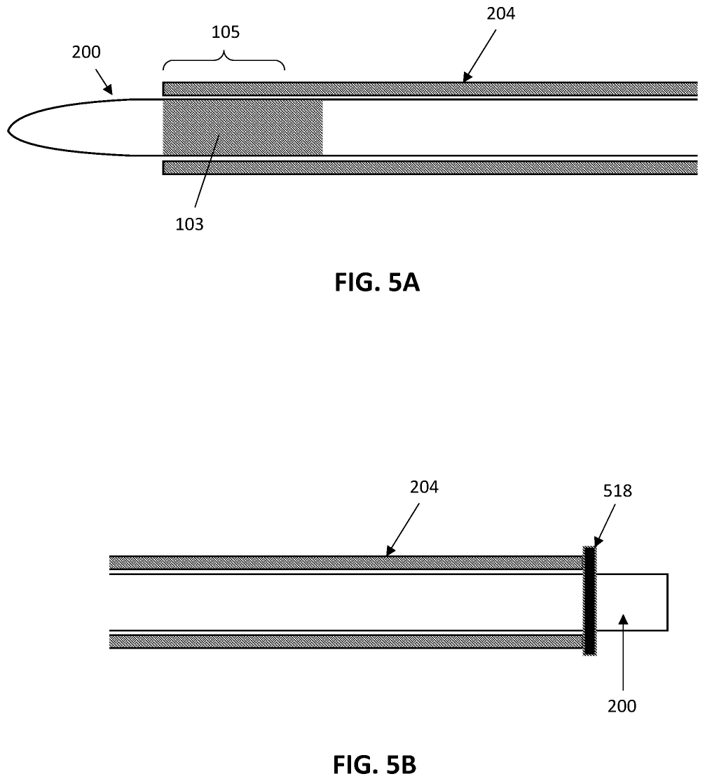

FIGS. 5A and 5B illustrate an example of an obturator 200 that is inserted into a catheter 204 so that the distal cannula engagement region 103 of the obturator is aligned with the distal end region of the catheter. The stiffness of the distal cannula engagement region 103 is matched to the stiffness of the distal end region of the catheter 105. FIG. 5B shows a proximal end of the obturator 200 and catheter 204, showing the two engaged with each other at the proximal end so that the distal cannula engagement region of the obturator is aligned with the distal end region of the cannula (or in some examples the distal cannula engagement region is slightly beyond (and spans) the distal end region of the catheter. In this example, a stop 518 on the obturator proximal end region may engage with the proximal end region of the catheter.

The distal cannula engagement region may be longer than the distal end region of the catheter, as shown in FIG. 5A. The length of the cannula and the length of the obturator may be selected so that the stop engages with the proximal end of the cannula when the distal cannula engagement region of the obturator is aligned with the distal end region as described above.

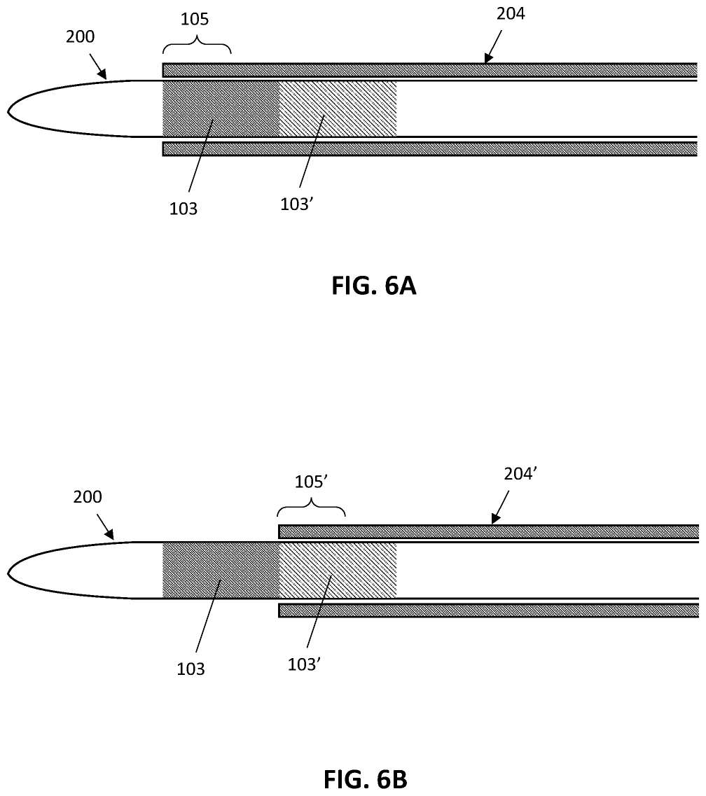

In some examples, such as the one shown in FIGS. 6A-6B, multiple, longitudinally separated distal cannula engagement regions may be included allowing the same obturator to be used with different catheter, which have different stiffness of their distal ends. For example, in FIG. 6A the obturator 200 includes a first distal cannula engagement region and a second distal cannula engagement region that are adjacent to each other. The first distal cannula engagement region and the second distal cannula engagement region may be separated from each other by a spacer distance (e.g., between 0.1 mm and 20 mm, between 0.1 mm and 10 mm, etc.). In FIG. 6A the first distal cannula engagement region is aligned with the distal end region 105 of a first cannula 204. The second distal cannula engagement region 103′ is shown proximally withdrawn into the first cannula. In FIG. 6B the same obturator 200 is shown engaged with a second cannula 204′ so that the second distal cannula engagement region of the obturator is aligned with the distal end region 105′ of the second cannula.

Throughout this description, the term obturator may apply to both an obturator and a dilator.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1.-20. (canceled)

21. A system comprising:
a catheter having a distal end region having a first stiffness; and
an obturator having a distal cannula engagement region having a stiffness that is matched to the first stiffness within +/−5% of the first stiffness, wherein the stiffness of the distal cannula engagement region is different from the stiffness of a tapered region distal to the distal cannula engagement region and is also different from a region of the obturator proximally adjacent to the distal cannula engagement region;
wherein the obturator and catheter are configured to engage to form a combined structure with each other proximally so that the distal cannula engagement region is aligned with the distal end region of the catheter to prevent forming a gap between the obturator and the catheter when the combined structure is bent or curved.

22. The system of claim 21, wherein the distal cannula engagement region comprises a stiffening component.

23. The system of claim 22, wherein the stiffening component comprises a tubular member within the distal cannula engagement region.

24. The system of claim 22, wherein the stiffening component comprises a material having a higher stiffness than a material forming the tapered region distal and/or the region of the obturator proximally adjacent to the distal cannula engagement region.

25. The system of claim 22, wherein the stiffening component comprises an internal frame.

26. The system of claim 21, wherein the distal cannula engagement region comprises a length that is between 1 mm and 40 mm.

27. The system of claim 21, wherein the distal cannula engagement region comprises a length that is greater than 5 mm.

28. The system of claim 21, wherein the obturator comprises an elongate guidewire channel extending down a length of the obturator.

29. The system of claim 21, wherein the distal cannula engagement region comprises a stiffening insert within the distal cannula engagement region.

30. An obturator comprising:
an elongate body comprising a tapered distal end and comprising a first material; and
a distal cannula engagement region of the elongate body, the distal cannula engagement region including an insert, wherein the distal cannula engagement region is proximal to the tapered distal end of the elongate body and distally adjacent to a second region of the elongate body, the insert comprising a material having a stiffness that is greater than the stiffness of the first material so that the stiffness of the distal cannula engagement region is greater than the stiffness of the tapered distal end and the second region.

31. The obturator of claim 30, further comprising an obturator lumen extending through the elongate body.

32. The obturator of claim 31, wherein the insert comprises an insert lumen coaxial with the obturator lumen.

33. The obturator of claim 30, wherein the insert comprises a length of about 1 mm-20 mm.

34. The obturator of claim 30, wherein the insert comprises a stiffness of between about 0.2 and about 2.5 GPa.

35. The obturator of claim 30, wherein a stiffness of the insert is greater than twice the stiffness of the first material.

36. The obturator of claim 30, wherein the insert is coaxial with the obturator.

37. The obturator of claim 30, wherein the obturator comprises an obturator or a dilator.

38. The obturator of claim 30, wherein the insert has an outer diameter that is less than the outer diameter of the distal cannula engagement region.

39. The obturator of claim 30, further comprising a stop on a proximal end region of the obturator configured to secure the distal cannula engagement region of the obturator in alignment with a distal end region of a catheter in which the obturator is inserted.
